(19) United States
(12) Patent Application Publication
(10) Pub. No.: US 2009/0004198 A1
(43) Pub. Date: Jan. 1, 2009

(54) Human monoclonal antibody binding to human cytomegalovirus and its antigen binding portion

(76) Inventors: Kantou Nakajima, Sapporo-shi (JP); Takeshi Matsumura, Sapporo-shi (JP); Uiko Kagaya, Sapporo-shi (JP)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/228,393

(22) Filed: Aug. 12, 2008

Related U.S. Application Data
(63) Continuation-in-part of application No. PCT/JP2007/052762, filed on Feb. 15, 2007.

(30) Foreign Application Priority Data
Feb. 15, 2006 (JP) 2006-037380

(51) Int. Cl. A61K 39/395; C07K 16/18; C12N 15/11; C12N 15/00; C12N 5/06; C12N 1/20; C12N 1/19; A61P 31/00

(52) U.S. Cl. 424/142.1; 530/388.15; 536/23.53; 435/320.1; 435/325; 435/252.3; 435/254.11

(57) ABSTRACT

The present invention aims to provide a human monoclonal antibody and an antigen binding portion of a human monoclonal antibody with higher affinity and neutralizing capacity to the human cytomegalovirus (HCMV), a virus which causes various diseases in situations where immunodeficiencies are present. The current invention provides an anti-human cytomegalovirus (HCMV) monoclonal antibody which is a human monoclonal antibody capable of binding to HCMV and neutralizing bioactivity of the HCMV, and which may be further characterized as possessing a light chain (L chain) comprising an amino acid sequence of SEQ ID. NO. 1, and has a heavy chain (H chain) comprising an amino acid sequence of SEQ ID NO. 2.

### RELATED APPLICATIONS

This application is a Continuation-In-Part Application of PCT/JP2007/052762, filed on Feb. 15, 2007, which claims priority to Japanese Patent Application No. 2006-037380, filed Feb. 15, 2006. The entire content and disclosure of the preceding applications are incorporated by reference into this application.

### BACKGROUND OF THE INVENTION

1. Field of the Invention

This invention relates to a human monoclonal antibody and its antigen binding portion binding to human Cytomegalovirus (described as “HCMV”).

2. Description of the Background Art

Human Cytomegalovirus (HCMV) belongs to the β-Herpesvirinae subfamily of the family Herpesviridae, including human herpes virus 6 (HHV-6) and human herpes virus 7 (HHV-7). HCMV is a double-stranded DNA virus, comprising 230 kbp coding more than 200 genes with the diameter of about 180 nm, and HCMV is the biggest virus among the family Herpesviridae.

HCMV exhibits strong species specificity. Humans can widely be infected with the HCMV that has affinity to widespread tissues in human body. However, no other animals than human is infected with this HCMV.

Once HCMV infects a human being, HCMV will remain forever with that host even though host immunity is acquired in the human body. HCMV maintains a latent infection in the human host's body during the host's lifetime once it infects. Although more than 90% of adult human are already infected with the HCMV and carry its virus, HCMV is harmless to healthy people.

However, the HCMV infects immunocompromised patients such as hemodialysis patients, cancer patients, patients who take immunosuppressants, HIV-carriers, bone-marrow transplant patients, and organ transplant patients with immunocompromised status; the HCMV is reactivated, and life-threatening diseases such as interstitial pneumonia, retinitis, gastroenteritis, and encephalitis develop.

Furthermore, when the HCMV infection is transmitted from pregnant women to their fetuses to cause cytomegalic inclusion disease, the infection leads to spontaneous abortion, stillbirth and death in the early years of life. Even though the fetus survives cytomegalic inclusion disease, the HCMV infection to the fetus may cause microcephaly, mental development disorder, mental retardation and hearing impairment.

The antiviral drug “ganciclovir” has been developed to suppress HCMV growth as a prophylactic agent or a therapeutic agent that can improve and alleviate life-threatening clinical conditions caused by said HCMV (AIDS/HIV Treatment Directory Vol. 5, No. 2 (Fall): American Foundation for AIDS Research (AmFAR): pp 43-45, 1991).

Ganciclovir is an antiviral drug used to block the synthesis of viral DNA by inhibiting DNA polymerase. Ganciclovir has been authorized, and is used to remedy cytomegalovirus retinitis of immunodeficient patients such as those with AIDS. It is also used to suppress symptoms of cytomegalovirus retinitis of the patients whose counts of CD4 lymphocyte are less than 100/mm³ because of progressed HIV infection.

However, it has been reported that ganciclovir has many kinds of side effects such as hematopoietic disorder, therefore its indication is particularly limited as the above description.

Bone marrow suppression is frequently reported as a side effect of ganciclovir which leads to blood disorders, therefore side effects must be paid attention to. White blood cell, red blood cell and blood platelet counts decrease in number. Then, fever, sore throat, lassitude, bleeding such as bleeding under the skin etc. are observed as early symptoms. In some cases, psychoneurotic disorder is observed and this causes headache, dizziness, insomnia, abnormality of thought and sense of insecurity etc.

Besides, since teratogenicity is reported in the experimental study using animals, ganciclovir may not be used to pregnant women.

Moreover, when the use of ganciclovir is applied in the severe cases of congenital HCMV infection, it is reported to reduce the number of expressed neurological sequelae effectively and to retard the progression of deafness effectively (Whitley R J, et al. Ganciclovir treatment of symptomatic congenital cytomegalovirus infection: results of a phase II study: National Institute of Allergy and Infections Disease Collaborative Antiviral Study Group, J Infect Dis., 1997: 175:1080-6). Therefore, the problems related to the side effect of ganciclovir, bone marrow suppression and infertility should be fully and carefully considered.

As per the above description, there has been a strong desire to develop a prophylactic or a therapeutic agent that can suppress or alleviate the life-threatening clinical conditions caused by said HCMV without any side effect.

Therefore, an antibody (hereinafter refer to “anti-HCMV antibody” in some cases) capable of binding to HCMV and neutralizing its infectiousness (lose biological activity) is expected to be a prophylactic or a therapeutic agent against life-threatening clinical condition caused by said HCMV. For example, the antibody may be used to prevent or treat different kinds of illness caused by HCMV, when the infected human is of an immunocompromised status.

In other words, it was considered effective to administer an anti-HCMV antibody that has strong affinity and neutralizing capacity against HCMV without any adverse reaction, namely an antibody drug, in order to suppress or alleviate symptom of illness by obliterating HCMV activity.

However, HCMV inhibitory antibodies reported so far (for instance, Japanese Patent Application Tokuhyo No. H8-502403, Japanese Patent Application Tokuhyo No. H8-506325) do not have sufficient affinity and neutralizing capacity against HCMV. Therefore, they were not sufficient to inhibit biological activity of natural HCMV well and to suppress or alleviate symptom of illness.

For this reason, it has been strongly desired to develop an anti-HCMV antibody and its antigen binding portion derived from human monoclonal antibody without immunological response, so that they may be applied to a prophylactic or a therapeutic agent with high affinity, specificity and neutralizing capacity against HCMV.

This invention focuses on providing a human monoclonal antibody and its antigen binding portion which excels in affinity and neutralizing capacity against HCMV causing various diseases for instance, in immunocompromised status.

### SUMMARY OF INVENTION

In an inventive approach, the inventors found that a human monoclonal antibody is sufficient in affinity and neutralizing capacity against HCMV. Then, the present invention was achieved.

In other words, one embodiment of the present invention relates to a human monoclonal antibody capable of binding to HCMV and neutralizing bioactivity of the HCMV, wherein the anti-HCMV monoclonal antibody has a light chain (L chain) comprising an amino acid sequence of sequence number 1, and has a heavy chain (H chain) comprising an amino acid sequence of sequence number 2.

Another embodiment of the present invention relates to a human monoclonal antibody or its antigen binding portion capable of binding to HCMV and neutralizing bioactivity of the HCMV, wherein the anti-HCMV monoclonal antibody or its antigen binding portion has a light chain variable region (LCVR) comprising an amino acid sequence of sequence number 3, and has a heavy chain variable region (HCVR) comprising an amino acid sequence of sequence number 4.

Yet another embodiment of the present invention relates to a human monoclonal antibody or its antigen binding portion capable of binding to HCMV and neutralizing bioactivity of the HCMV, wherein the anti-HCMV monoclonal antibody or its antigen binding portion has the following complementarity-determining region (CDR) domains:
(a) a light chain (L chain) CDR 1 domain having an amino acid sequence of sequence number 5,
(b) a light chain (L chain) CDR 2 domain having an amino acid sequence of sequence number 6, and
(c) a light chain (L chain) CDR 3 domain having an amino acid sequence of sequence number 7.
(d) a heavy chain (H chain) CDR 1 domain having an amino acid sequence of sequence number 8,
(e) a heavy chain (H chain) CDR 2 domain having an amino acid sequence of sequence number 9, and
(f) a heavy chain (H chain) CDR 3 domain having an amino acid sequence of sequence number 10.

Yet another embodiment of the present invention relates to the anti-HCMV monoclonal antibody or its antigen binding portion, wherein, in the complementarity-determining region (CDR), the anti-HCMV monoclonal antibody or its antigen binding portion has an amino acid sequence which has deletion, substitution, insertion or addition of one or several amino acid residues, and approximately 1 μg/mL of the anti-HCMV monoclonal antibody or its antigen binding portion has a blocking rate of HCMV infection over 80%.

Yet another embodiment of the present invention relates to the anti-HCMV monoclonal antibody or its antigen binding portion, wherein the anti-HCMV monoclonal antibody or its antigen binding portion has an affinity (M) from Kd=1.0×10⁻¹¹ (M) to 1.3×10⁻¹¹ (M).

Yet another embodiment of the present invention relates to the anti-HCMV monoclonal antibody or its antigen binding portion, wherein approximately 1 μg/mL of the anti-HCMV monoclonal antibody or its antigen binding portion has a blocking rate of HCMV infection over 80%.

Yet another embodiment of the present invention relates to the anti-HCMV monoclonal antibody or its antigen binding portion, wherein the antibody belongs to the IgG₁ (K) class (subclass).

Yet another embodiment of the present invention relates to a prophylactic or therapeutic agent for disease caused by the HCMV comprising the anti-HCMV monoclonal antibody or its antigen binding portion.

Yet another embodiment of the present invention relates to the prophylactic or therapeutic agent for disease caused by the HCMV comprising the anti-HCMV monoclonal antibody or its antigen binding portion, wherein the disease caused by the HCMV is selected from any one of the following groups:
(a) interstitial pneumonia, retinitis, gastroenteritis and encephalitis with reactivation of HCMV in immunodeficiency state,
(b) cytomegalic inclusion disease caused by HCMV infection from pregnant mother to fetus,
(c) death due to spontaneous abortion and stillbirth caused by the cytomegalic inclusion disease, and death in the early years of life caused by the cytomegalic inclusion disease,
(d) when survived in the case of (c), microcephaly, mental development disorder, mental retardation and hearing impairment caused by the cytomegalic inclusion disease.

Yet another embodiment of the present invention relates to an isolated deoxyribonucleic acid (DNA) coding for an anti-HCMV monoclonal antibody or its antigen binding portion capable of binding to HCMV and neutralizing bioactivity of the HCMV, wherein the isolated DNA codes for any one of amino acid sequences of the sequence numbers 1 to 4, three amino acid sequences of the sequence numbers 5 to 7, or three amino acid sequences of the sequence numbers 8 to 10.

Yet another embodiment of the present invention relates to an isolated DNA capable of hybridizing with the DNA under stringent conditions.

Yet another embodiment of the present invention relates to a vector other than a plant expression vector, comprising the isolated DNA.

Yet another embodiment of the present invention relates to a host cell other than a plant cell, comprising the vector, wherein the isolated DNA is integrated into the vector.

An anti-HCMV antibody or its antigen binding portion of the present invention specifically binds to HCMV causing various diseases, and obliterate (neutralize) a bioactivity, so that the anti-HCMV antibody or its antigen binding portion may express high affinity and high neutralizing capacity (to HCMV). The anti-HCMV antibody or its antigen binding portion is derived from human monoclonal antibody. Therefore, the anti-HCMV antibody or its antigen binding portion does not show immunogenicity and rejection.

According to these properties, the anti-HCMV antibody or its antigen binding portion of the present invention shows high affinity, high neutralizing capacity to HCMV, and also no rejection. Therefore, the anti-HCMV antibody or its antigen binding portion is thought to be effective as a prophylactic or a therapeutic agent for various diseases resulting from HCMV including (a) life-threatening diseases such as interstitial pneumonia, retinitis, gastroenteritis and encephalitis caused by reactivation of HCMV in immunodeficiency state such as AIDS, cancer, and after organ transplantation, bone marrow transplantation and hemodialysis, (b) cytomegalic inclusion disease caused by HCMV infection from pregnant mother to fetus, (c) death due to spontaneous abortion and stillbirth caused by the cytomegalic inclusion disease, and death in the early years of life caused by the cytomegalic inclusion disease, (d) when survived in the case of (c), microcephaly, mental development disorder, mental retardation and hearing impairment caused by the cytomegalic inclusion disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The anti-HCMV monoclonal antibody or its antigen binding portion according to the present invention will be described below.

FIG. 1 is a flow chart showing the procedure for separating antibody-producing cell clone of the anti-HCMV antibody according to the present invention.

FIG. 2 shows the ELISA result indicating the anti-HCMV antibody isotype according to the present invention.

FIG. 3 shows the SDS-PAGE result of anti-HCMV antibody according to the present invention.

FIG. 4 shows the Western blot result of antigen specificity of anti-HCMV antibody according to the present invention.

FIG. 5 shows the neutralizing activity of anti-HCMV antibody according to the present invention.

FIG. 6 shows the Western blot result of an anti-HCMV antibody production caused by introducing anti-HCMV antibody gene to 293T cell.

FIG. 7 shows the ELISA result indicating an anti-HCMV antibody production caused by introducing anti-HCMV antibody gene to 293T cell.

### DETAILED DESCRIPTION

As used herein, the term “antibody” indicates not only immunoglobulin molecule interactively connected with 4 polypeptide chains, 2 heavy chains (H) and 2 light chains (L) with disulfide bond in the molecule, but also immunoglobulin molecule interactively connected with 2 polypeptide chains, 1 heavy chain (H) and 1 light chain (L) with disulfide bond in the molecule.

The term “antigen binding portion” of the antibody (or simply antigen portion) indicates one or plural antibody fragments with a specific antigen binding activity (HCMV, for example).

The term “neutralizing antibody against the HCMV activity” indicates the antibody inhibiting bioactivity of the HCMV by binding to the HCMV.

The terms such as “inhibitory effect”, “inhibition”, and “inhibiting” mean 5 to 100% depression of bioactivity caused by a natural HCMV antigen, whatever they are derived from. For therapeutic purposes, ideally, the inhibition of HCMV bioactivity should be between 50% and 100%.

One aspect of the present invention relates to the human monoclonal antibody capable of binding to HCMV and neutralizing bioactivity of the HCMV, characterized in that the anti-HCMV monoclonal antibody or its antigen binding portion has a light chain (L chain) comprising an amino acid sequence of sequence number 1, and has a heavy chain (H chain) comprising an amino acid sequence of sequence number 2.

When the human monoclonal antibody or its antigen binding portion binds specifically to the HCMV and neutralizes its bioactivity, the following properties of the anti-HCMV monoclonal antibody and its antigen binding portion are also incorporated into the present invention:

Anti-HCMV monoclonal antibody and its antigen binding portion, characterized in that, in the light chain (L chain), the anti-HCMV monoclonal antibody has an amino acid sequence comprising sequence number 1, from which not less than one amino acid is deleted, with which not less than one amino acid is substituted, or to which not less than one amino acid is inserted or added;

Anti-HCMV monoclonal antibody, characterized in that, in the heavy chain (H chain), the anti-HCMV monoclonal antibody has an amino acid sequence comprising sequence number 2, from which not less than one amino acid is deleted, with which not less than one amino acid is substituted, or to which not less than one amino acid is inserted or added.

Each of the heavy chains includes the heavy chain variable region (suitably abbreviated to “HCVR” or “V_{H}”) and the heavy chain constant region (the 3 domains, which is abbreviated to “C_{H}1”, “C_{H}2”, and “C_{H}3”).

Each of the light chains includes the light chain variable region (suitably abbreviated to “LCVR” or “V_{L}”) and the light chain constant region (a domain abbreviated to “C_{L}”). The HCVR and LCVR are important for the binding specificity of the antibody.

Another aspect of the present invention relates to the human monoclonal antibody or its antigen binding portion capable of binding to HCMV and neutralizing bioactivity of the HCMV, characterized in that the anti-HCMV monoclonal antibody or its antigen binding portion has a light chain variable region (LCVR) comprising an amino acid sequence of sequence number 3, and has a heavy chain variable region (HCVR) comprising an amino acid sequence of sequence number 4.

When the human monoclonal antibody or its antigen binding portion binds specifically to the HCMV and neutralizes its bioactivity, the following properties of the anti-HCMV monoclonal antibody and its antigen binding portion are also incorporated into the present invention:

Anti-HCMV monoclonal antibody or its antigen binding portion, characterized in that, in the light chain variable region (LCVR) and the heavy chain variable region (HCVR), the anti-HCMV monoclonal antibody or its antigen binding portion has the amino acid sequence, from which not less than one amino acid is deleted, with which not less than one amino acid is substituted, or to which not less than one amino acid is inserted or added;

Anti-HCMV monoclonal antibody or its antigen binding portion, characterized in that, in either the light chain variable region (LCVR), or the heavy chain variable region (HCVR), the anti-HCMV monoclonal antibody or its antigen binding portion has the amino acid sequence, from which not less than one amino acid is deleted, with which not less than one amino acid is substituted, or to which not less than one amino acid is inserted or added.

The amino acid sequence of the variable region almost rules interaction between the antibody and antigen. Therefore, when an expression vector containing a variable region sequence derived from a natural antibody integrated in a framework sequence derived from a different antibody with a property is constructed, the resulting vector can express a recombinant antibody with a property of the natural antibody.

Therefore, when an intact recombinant antibody having the same binding property with the original antibody is generated, it is unnecessary to use a complete sequence of the original antibody. The sequence of the heavy chain and the light chain including the variable region achieves that purpose well.

When the human monoclonal antibody or its antigen binding portion binds specifically to the HCMV and neutralizes its bioactivity, the following properties of the anti-HCMV monoclonal antibody and its antigen binding portion are also incorporated into the present invention:

The anti-HCMV monoclonal antibody and its antigen binding portion having a variable region comprising number 3 or 4 amino acid sequence;

The anti-HCMV antibody and its antigen binding portion, characterized in that, in the variable region, anti-HCMV monoclonal antibody or its antigen binding portion has the amino acid sequence, from which not less than one amino acid is deleted, with which not less than one amino acid is substituted, or to which not less than one amino acid is inserted or added.

The antibody mainly interacts with the target antigen through the amino acid residue on the LCVR and HCVR. Therefore, in the variable regions, there are more various amino acid sequence than those outside the variable region.

The HCVR and LCVR are further subdivided into the invariable “framework region (FR)” and the hyper-variable “complementarity determining region (CDR)”. The HCVR and LCVR consist of the 3 CDRs and 4 FRs, respectively. Their order is FR1, CDR1, FR2, CDR2, FR3, CDR3, and FR4, from the amino-terminal to the carboxy-terminal.

Another aspect of the present invention relates to the human monoclonal antibody or its antigen binding portion capable of binding to HCMV and neutralizing bioactivity of the HCMV, characterized in that the anti-HCMV monoclonal antibody or its antigen binding portion has a complementarity-determining region (CDR) coded for at least one amino acid sequence selected from the group of sequence numbers 5 to 10.

When the human monoclonal antibody or its antigen binding portion binds specifically to the HCMV and neutralizes its bioactivity, the following properties of the anti-HCMV monoclonal antibody or its antigen binding portion is also incorporated into the present invention: anti-HCMV monoclonal antibody or its antigen binding portion, characterized in that, in the light chain of the complementarity determining region (CDR), the anti-HCMV monoclonal antibody or its antigen biding portion has the amino acid sequence from which not less than one amino acid is deleted, with which not less than one amino acid is substituted, or to which not less than one amino acid is inserted or added.

Another aspect of the present invention relates to the human monoclonal antibody or its antigen binding portion capable of binding to HCMV and neutralizing bioactivity of the HCMV, characterized in that the anti-HCMV monoclonal antibody or its antigen binding portion has CDR domain selected from the following groups, the groups comprising:
(a) a light chain (L chain) CDR 1 domain having an amino acid sequence of sequence number 5,
(b) a light chain (L chain) CDR 2 domain having an amino acid sequence of sequence number 6, and
(c) a light chain (L chain) CDR 3 domain having an amino acid sequence of sequence number 7.

When the human monoclonal antibody or its antigen binding portion binds specifically to the HCMV and neutralizes its bioactivity, the following properties of the anti-HCMV monoclonal antibody or its antigen binding portion is also incorporated into the present invention: Anti-HCMV monoclonal antibody or its antigen binding portion, characterized in that, in the heavy chain of the complementarity-determining region (CDR), the anti-HCMV monoclonal antibody or its antigen binding portion has the amino acid sequence from which not less than one amino acid is deleted, with which not less than one amino acid is substituted, or to which not less than one amino acid is inserted or added.

Another aspect of the present invention relates to the human monoclonal antibody or its antigen binding portion capable of binding to HCMV and neutralizing bioactivity of the HCMV, characterized in that the anti-HCMV monoclonal antibody or its antigen binding portion has a CDR domain selected from the following groups, the groups comprising:
(a) a heavy chain (H chain) CDR 1 domain having an amino acid sequence of sequence number 8,
(b) a heavy chain (H chain) CDR 2 domain having an amino acid sequence of sequence number 9, and
(c) a heavy chain (H chain) CDR 3 domain having an amino acid sequence of sequence number 10.

When the human monoclonal antibody or its antigen binding portion binds specifically to the HCMV and neutralizes its bioactivity, the following properties of the anti-HCMV monoclonal antibody and its antigen binding portion are also incorporated into the present invention:

anti-HCMV monoclonal antibody and its antigen binding portion, characterized in that, in the heavy chain of the complementarity determining region (CDR), the anti-HCMV monoclonal antibody or its antigen binding portion has the amino acid sequence from which not less than one amino acid is deleted, with which not less than one amino acid is substituted, or to which not less than one amino acid is inserted or added.

According to the present invention, the anti-HCMV monoclonal antibody or its antigen binding portion which can bind specifically with a particular antigenic determinant (epitope) and neutralizes the bioactivity.

The variation of the anti-HCMV monoclonal antibody or its antigen binding portion is explained as the above description, but the described features may be substituted or converted in the technology field without departing from the scope of the invention.

Namely, in the scope of the present invention, the following antibodies or its antigen binding portions are included:
I. the full length antibody
II. the antigen binding portion in I
III. the recombinant human monoclonal antibody capable of specifically binding to HCMV and neutralizing bioactivity, characterized in that the recombinant human monoclonal antibody is obtained by any well-known techniques, applying the variable regions and CDRs comprising the amino acid sequence numbers 3-10, and
IV. the recombinant human monoclonal antigen binding portion in III.

For example, the leader sequences of the heavy chain and light chain are cleaved in the protein maturation process. The cleaved leader sequences have no effect on the final antibody properties. To complement the cleaved sequence, the cloned cDNA is integrated with the synthetic oligonucleotide in ligation or PCR amplification method.

In an alternative process, a whole variable region is synthesized with a pair of short overlapping oligonucleotide, and then the oligonucleotide is amplified in a PCR amplification method, so that an artificial clone of a variable region is entirely obtained.

Another aspect of the present invention relates to an isolated Deoxyribonucleic acid (DNA) coding the anti-HCMV monoclonal antibody or its antigen binding portion capable of binding to HCMV and neutralizing bioactivity of the HCMV, characterized in that the isolated DNA codes for at least one amino acid sequence selected from the group of the sequence numbers 1 to 10.

When an isolated Deoxyribonucleic acid (DNA) codes for the anti-HCMV monoclonal antibody or its antigen binding portion capable of binding to HCMV and neutralizing bioactivity of the HCMV, the following properties of the isolated DNA is also incorporated into the present invention: the isolated DNA capable of hybridizing with the DNA described above under stringent conditions.

Vector and host cell are also incorporated in the present invention below.

1) A vector incorporating the isolated DNA mentioned above.
2) A host cell integrated with the expression vector mentioned above.

These recombinant expression vector and host cell are preferably selected from animal cell in particularly mammalian cell. Plant expression vector and plant cell are avoided, because recombinant expression vector and host cell of the present invention are aimed to express recombinant human antibody.

Furthermore, applying genetic engineering techniques, a phage display method is developed. The phage display method is applied to express a recombinant antibody on the phage surface. Also, the phage display method is applied to artificially shuffle V_{H} and V_{L} genes for preparing a diversified scFv (single chain Fragment of variable region) antibody. It is also possible to obtain a specific antibody by expressing the scFv antibody as phage fusion protein.

This is highly evaluated as a human antigen construction method applied for avoiding immunity and cell fusion technique. When a specific antibody or its antigen binding portion produced by the above method is applied based on the amino acid sequence number 1-10 herein, the antibody or its antigen binding portion is also incorporated in the present invention.

The antibody or its antigen binding portion may specifically bind to HCMV causing various diseases in immunodeficient condition, and may neutralize the HCMV bioactivity. Especially, the anti-HCMV antibody or its antigen binding portion in the present invention is characterized in its high affinity and high neutralizing activity to the HCMV.

A term “Specific binding” means that the antibody binds to a certain antigen. Generally, antibody binds to antigen at the least affinity of about 1×10⁻⁷ (M), and binds to a certain antigen with high affinity at least twice in comparison with the nonspecific antigen (e.g. BSA, casein).

A term “High affinity” to a certain IgG antibody means an antibody has a binding affinity at least about 1×10⁻⁷ (M), preferably at least about 1×10⁻⁸ (M), more preferably at least 1×10⁻⁹ (M), and much more preferably at least 1×10⁻¹⁰ (M). However, definition of “high affinity” is different among the antibody isotypes. For example, the IgM isotype is defined to have a “high affinity”, when it has a binding affinity at least 1×10⁻⁷ (M).

Affinity of the anti-HCMV antibody or of its antigen binding portion in the present invention usually has dissociation constant of K_{d}=1.0×10⁻¹¹ (M) ˜1.3×10⁻¹¹ (M).

Approximately 1 μg/mL of the anti-HCMV monoclonal antibody or its antigen binding portion has an infection blocking rate of over 80%, which means sufficient neutralizing capacity.

Another aspect of present invention relating to the anti-HCMV monoclonal antibody, is characterized in that the anti-HCMV monoclonal antibody belongs to the IgG₁, (k).

It is known that the IgG antibody has a high affinity and stability. On the other hand, IgM antibody is known to aggregate itself rapidly and to have a low affinity.

The anti-HCMV antibody or its antigen binding portion in the present invention has high affinity and neutralizing capacity to the HCMV. Therefore, it is expected to be applied as prophylactic or therapeutic agent for the various diseases caused by the HCMV, for example (a) life-threatening disease such as interstitial pneumonia, retinitis, gastroenteritis and encephalitis with reactivation of HCMV in immunodeficiency state such as AIDS, cancer, and after organ transplantation, bone marrow transplantation and hemodialysis, (b) cytomegalic inclusion disease caused by HCMV infection from pregnant mother to fetus, (c) death due to spontaneous abortion and stillbirth caused by the cytomegalic inclusion disease, and death in the early years of life caused by the cytomegalic inclusion disease, (d) when survived in the case of (c), microcephaly, mental development disorder, mental retardation and hearing impairment caused by the cytomegalic inclusion disease.

As used herein, the term of “a disease caused by HCMV” includes any diseases that cause and worsen the pathophysiology, when a subject has a HCMV. The term also includes other diseases that cause and worsen the pathophysiology.

It is expected that inhibiting the biological activity of HCMV may palliate the disease symptoms caused by HCMV, and/or may palliate the disease progression.

For example, HCMV may be detected in the biological fluid by applying the anti-HCMV antibody. By increasing a HCMV concentration in the subject's biological fluid, it is possible to assess whether the subject suffers from the disease or not (for example, increasing HCMV concentration in blood serum, blood plasma, and synovial fluid in the subject).

Furthermore, anti-HCMV monoclonal antibody or its antigen biding portion in the present invention is obtained from an antibody-producing cell proliferated by EVB infection after removal of T-cell from mononuclear cell isolated from human blood which the anti-HCMV antibody in serum gives positive result. Accordingly, the anti-HCMV monoclonal antibody or its antigen binding portion in the present invention is a complete human monoclonal antibody.

Therefore, it does not show immunogenicity or rejection response, when it is administrated to human body as the antibody preparation aimed to inhibit lethal disease onset with HCMV or to reduce pathology with HCMV.

### EXAMPLES

Hereinafter, examples of the present invention will be described more specifically, but the examples do not limit the scope of the present invention.

Isolation of a Cell Clone Producing a HCMV Antibody.

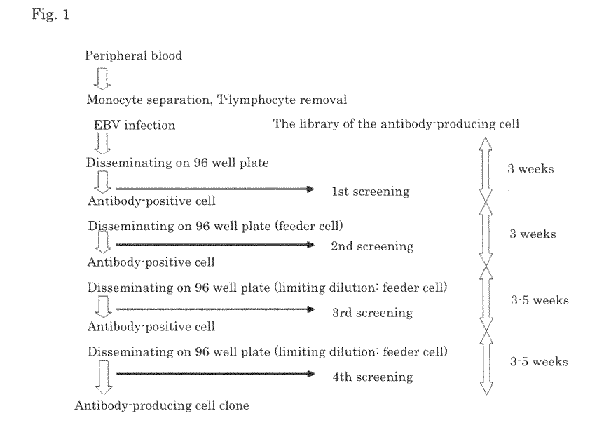

FIG. 1 is a flow chart to isolate antibody producing cell clones. Mononuclear cells were separated from human blood having high anti-HCMV antibody titer in the serum.

T-cells were removed from mononuclear cells. The remaining cells were infected with EBV. Thereafter, the cells which started to proliferate were used as a cell library.

The antibody-producing cell library were plated in 96 well plate. After about 3-4 weeks cultivation, 1st screening to detect anti-HCMV antibody was conducted in the supernatant solution.

The ELISA method was applied to screen the antibody against AD1 which is one of major neutralizing epitope of HCMV, in the 96 well plate coated with GST fused HCMV-AD1.

The obtained population of antibody-positive cells was plated in another 96 well plate in a low density, and then feeder cells were added to each well to accelerate cell proliferation.

After 3-4 weeks cultivation, 2nd screening for anti-HCMV antibody was conducted.

The obtained population of antibody-positive cells was plated in another 96 well plate by limiting dilution. In this step, feeder cells were also added to the cell population to accelerate its proliferation.

After 3-5 weeks cultivation, 3rd screening for anti-HCMV antibody was conducted.

The obtained clones of antibody-positive cells were plated by limiting dilution, and screened. Finally an antibody-positive cell clone was obtained in a well where one cell was originally plated.

2. Identification of Antibody Isotype.

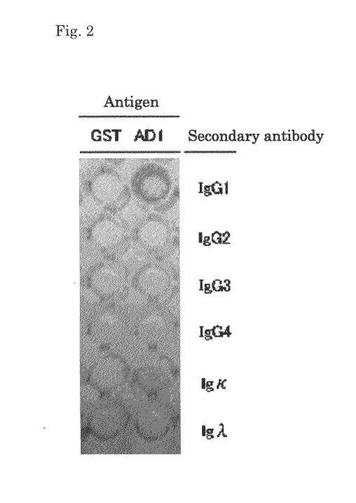

The isotype of the antibody, which was produced by isolated antibody-positive cell clone, was identified by ELISA method as used in the screening. Specific antibodies against isotype were applied as a second antibody. And then, a sample of supernatant of cell culture was tested. FIG. 2 showed that an obtained anti-HCMV antibody belongs to IgG1 kappa.

3. Purification of Antibody.

Purification of an anti-HCMV antibody was conducted by affinity purification method using Protein A. HiTrap rProtain A FF (Amersham) was applied as a prepacked column, and purification was carried out based on the condition recommended by column maker.

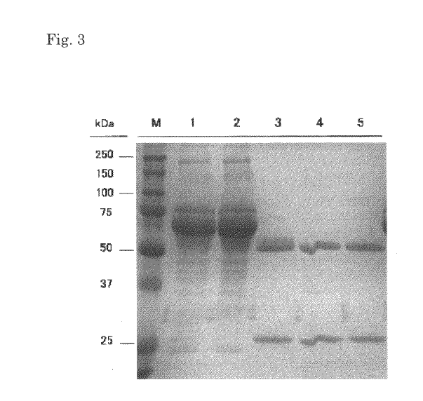

FIG. 3 shows the result of SDS-PAGE.

After the purification of the antibody, H chain of 50 kDa and L chain of 25 kDa were observed.

After purification of the antibody, it was confirmed by ELISA that the antibody preserved anti-HCMV activity.

4. Identification of Antigen Specificity of Anti-HCMV Antibody.

In the screening of anti-HCMV antibody, only epitope portion of the HCMV was fused with GST and used as an antigen. Therefore, it is necessary to confirm antibody specificity against whole HCMV antigen. Then, specific binding of the purified anti-HCMV antibody to the HCMV-AD1 was confirmed by the Western blot.

Glycoprotein B (gB), which is a major envelop protein of HCMV, is formed of AD-1 and AD-2.

Since HCMV-infected cells express gB on the cell membrane, HCMV-infected cells were prepared as sample. Human embryonic lung fibroblast (HEL) was chosen and cultivated for 2 days after HCMV (AD169 strain) infection. The lysate of this HCMV-infected cells was applied to SDS-PAGE, and blotted on the nitrocellulose membrane. The purified HCMV antibody and peroxidase-labelled anti-human IgG antibody (Medical & Biological Laboratories Co., LTD) were used as primary and secondary antibodies, respectively.

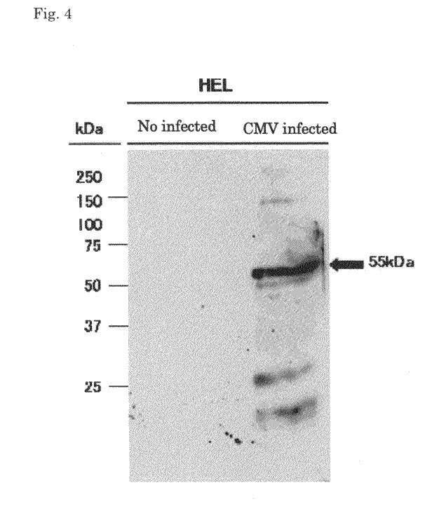

ECL reagent (Amersham Biosciences) was used for detection purpose. FIG. 4 is the result obtained by the Western blot, which shows antigen specificity of the anti-HCMV antibody.

No band was detected in the uninfected HEL cells, however, the specific band was detected in the HCMV infected HEL cells at molecular mass of 55 kDa, which is equivalent to that of the HCMV-AD1.

These results indicate that the anti-HCMV antibody (G3D No. 9) has specificity to the HCMV-AD1.

5. Affinity Analysis of Anti-HCMV Antibody.

Affinity analysis of the anti-HCMV antibody was requested to BIAcore. Analysis was carried out as follows: the antigen was fixed to a sensor chip, an antibody was added to the surface of the fixed antigen, and then the interaction between antibody and an antigen was measured.

As the antigen, GST-HCMV-AD1 fusion protein was used and as the control, GST protein was used.

The result indicated that the affinity of the antibody had dissociation constant of 1.16×10⁻¹¹ (M).

6. Evaluation on Neutralizing Activity of the Anti-HCMV Antibody.

A neutralizing activity was examined to evaluate the effectiveness of the anti-HCMV antibody (G3D No. 9). The neutralizing activity of the antibody was evaluated with the inhibition rate of HCMV (AD169 strain) infection to the HEL (human embryonic lung fibroblast) cells. In this assay, a virus suspension, a purified antibody (G3D No. 9) and a complement (5%) were mixed, incubated at 37° C. for 1 hour, and then infected to a HEL cell at 37° C. for 1 hour.

The cells were washed, cultivated for 2 days, and immunostained to detect pp 65, which is an early protein expressed by HCMV infection.

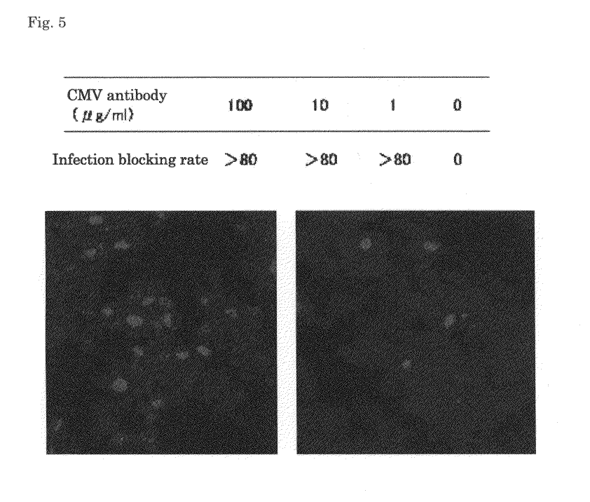

FIG. 5 shows the neutralizing activity of the anti-HCMV antibody. The G3D No. 9 antibody showed more than 80% of infection inhibition rate at the concentration of 1 ug/ml of the antibody, indicating that the antibody has effective neutralizing activity.

7. Cloning of antibody gene from antibody-producing cells.

Antibody genes were cloned by PCR method. Total-RNA was extracted from an anti-HCMV antibody-producing cell clone. A cDNA was synthesized with Oligo-dT primer and reverse transcriptase using the total-RNA as a template.

In both H-chain and L-chain, their antibody genes code for the variable regions on their 5′-portion, and their sequences vary among different antibodies. 3′-portion of their antibody genes code for constant regions which are conserved among the antibodies genes. Based on a nucleotide sequence database of antibodies genes, primers described in Table 1 were designed to contain an initiation site on the 5′-end, and a termination site on the 3′-end. And then, antibody gene was amplified by using the reverse-transcribed RNA as a template.

TABLE 1

light chain:
Light-kppa FW primer
(5′- CACCTTCTCACAATGAGGCTCC -3′)
Light-kappa RV primer
(5′- CACTTCTCCCTCTAACACTCTCC -3′)
heavy chain:
Heavy-gamma FW primer
(5′- GAACACAGAGAACTCACCATGGA -3′)
Heavy-gamma RV primer
(5′- GCCGTCGCACTCATTTACCCGGA -3′)

8. Determination of amino acid sequence based on antibody DNA sequence.

Nucleotide sequence of antibody DNA was determined using ABI sequencer. Obtained nucleotide sequences of anti-HCMV antibody DNA are shown in sequence numbers 1 through 10.

9. Confirmation that obtained antibody DNA encodes anti-HCMV antibody.

The obtained cDNA clones (H-chain and L-chain) were inserted into plasmid vector pSG5, respectively. Both plasmids were introduced into 293T cells, and transient expression of the antibody was confirmed. For transfection of plasmids, Lipofectamine (Invitrogen Co., LTD) and Plus reagent (Invitrogen Co., LTD) were used. pEGFP was transfected as a control, and transfection efficiency was confirmed by observing cells expressing GFP under a fluorescence microscope. The efficiency was 50% to 60%.

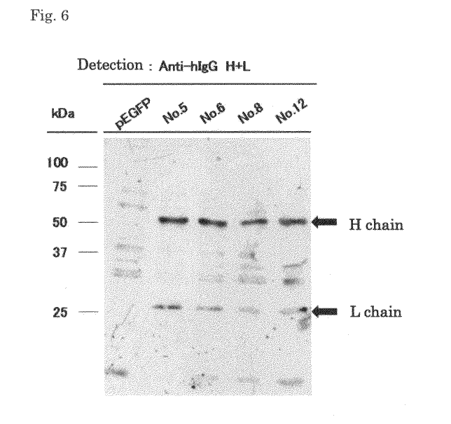

FIG. 6 is a Western blot analysis which detected a human antibody using the lysate of 293T cells 2 days after transfection.

For detection of antibody protein, peroxidase-labeled anti-human IgG H+L (Amersham) was applied, and both heavy (H) and light (L) chains were detected in one step. H-chain (50 kDa) and L-chain (25 kDa) in human antibody were detected in the transformed cell (FIG. 6).

Next, the same transfection experiment was conducted. In addition to the antibody secretion, preservation of the antigen specificity was confirmed.

Cell culture supernatants were obtained 24 hours and 48 hours after transfection. Human IgG antibody and HCMV specific antibody were detected by ELISA.

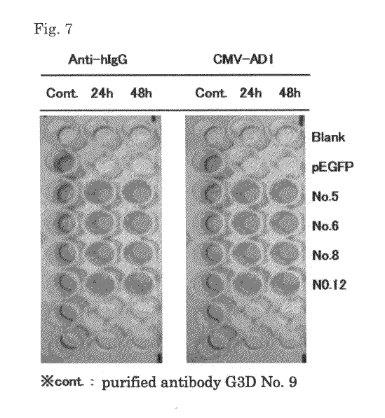

FIG. 7 shows the result of ELISA confirming that anti-HCMV antibody was secreted by 293T cells after transfection of anti-HCMV antibody gene to the cells.

Dilution series of the purified antibody was used as a control in ELISA.

pEGFP-transformed cell culture supernatant was applied as a control for transfection experiment as mentioned above. Human IgG was detected in each culture supernatant of the cells expressing antibody gene transiently. It was confirmed that all of secreted antibodies have the specificity against HCMV (FIG. 7).

Anti-HCMV antibody or its antigen binding portion obtained as above is able to specifically bind to HCMV causing various diseases, and is able to depress (neutralize) the bioactivity, so that affinity and neutralizing capacity (against HCMV) are shown to be excellent. Because the anti-HCMV monoclonal antibody is of human origin, the antibody shows no immunogenicity and does not evoke rejection reaction.

Considering these properties, the anti-HCMV antibody or its antigen binding portion according to the present invention is effective to be applied as prophylactic or therapeutic agent for the various diseases caused by HCMV, for example (a) life-threatening diseases such as interstitial pneumonia, retinitis, gastroenteritis and encephalitis with reactivation of HCMV in immunodeficiency state such as AIDS, cancer, and after organ transplantation, bone marrow transplantation and hemodialysis, (b) cytomegalic inclusion disease caused by HCMV infection from pregnant mother to fetus, (c) death due to abortion and stillbirth caused by the cytomegalic inclusion disease, and death in the early years of life caused by the cytomegalic inclusion disease, (d) when survived in the case of (c), microcephaly, mental development disorder, mental retardation and hearing impairment caused by the cytomegalic inclusion disease.

What is claimed is:

1. A human monoclonal antibody capable of binding to HCMV and neutralizing bioactivity of the HCMV, wherein the anti-HCMV monoclonal antibody has a light chain (L chain) comprising an amino acid sequence of SEQ ID NO. 1, and has a heavy chain (H chain) comprising an amino acid sequence of SEQ ID NO. 2.

2. A human monoclonal antibody or its antigen binding portion capable of binding to HCMV and neutralizing bioactivity of the HCMV, wherein the anti-HCMV monoclonal antibody or its antigen binding portion has a light chain variable region (LCVR) comprising an amino acid sequence of SEQ ID NO. 3, and has a heavy chain variable region (HCVR) comprising an amino acid sequence of SEQ ID NO. 4.

3. A human monoclonal antibody or its antigen binding portion capable of binding to HCMV and neutralizing bioactivity of the HCMV, wherein the anti-HCMV monoclonal antibody or its antigen binding portion has the following complementarity-determining region (CDR) domains:
(a) a light chain (L chain) CDR 1 domain having an amino acid sequence of SEQ ID NO. 5,
(b) a light chain (L chain) CDR 2 domain having an amino acid sequence of SEQ ID NO. 6,
(c) a light chain (L chain) CDR 3 domain having an amino acid sequence of SEQ ID NO. 7,
(d) a heavy chain (H chain) CDR 1 domain having an amino acid sequence of SEQ ID NO. 8,
(e) a heavy chain (H chain) CDR 2 domain having an amino acid sequence of SEQ ID NO. 9, and
(f) a heavy chain (H chain) CDR 3 domain having an amino acid sequence of SEQ ID NO. 10.

4. The anti-HCMV monoclonal antibody or its antigen binding portion according to claim 3, wherein, in any one of the complementarity-determining regions (CDR), the anti-HCMV monoclonal antibody or its antigen binding portion has an amino acid sequence which has deletion, substitution, insertion and/or addition of one or several amino acid residues, and approximately 1 μg/mL of the anti-HCMV monoclonal antibody or its antigen binding portion has a blocking rate of HCMV infection over 80 W.

5. The anti-HCMV monoclonal antibody according to claim 1, wherein the anti-HCMV monoclonal antibody has an affinity (M) from Kd=1.0×10⁻¹¹ (M) to 1.3×10⁻¹¹ (M).

6. The anti-HCMV monoclonal antibody or its antigen binding portion according to claim 2, wherein the anti-HCMV monoclonal antibody or its antigen binding portion has an affinity (M) from Kd=1.0×10⁻¹¹ (M) to 1.3×10⁻¹¹ (M).

7. The anti-HCMV monoclonal antibody or its antigen binding portion according to claim 3, wherein the anti-HCMV monoclonal antibody or its antigen binding portion has an affinity (M) from Kd=1.0×10⁻¹¹ (M) to 1.3×10⁻¹¹ (M).

8. The anti-HCMV monoclonal antibody according to claim 1, wherein approximately 1 μg/mL of the anti-HCMV monoclonal antibody has a blocking rate of HCMV infection over 80%.

9. The anti-HCMV monoclonal antibody or its antigen binding portion according to claim 2, wherein approximately 1 μg/mL of the anti-HCMV monoclonal antibody or its antigen binding portion has a blocking rate of HCMV infection over 80%.

10. The anti-HCMV monoclonal antibody or its antigen binding portion according to claim 3, wherein approximately 1 μ/mL of the anti-HCMV monoclonal antibody or its antigen binding portion has a blocking rate of HCMV infection over 80%.

11. The anti-HCMV monoclonal antibody according to claim 1, wherein the antibody belongs to the IgG₁ (κ) class (subclass).

12. The anti-HCMV monoclonal antibody or its antigen binding portion according to claim 2, wherein the antibody belongs to the IgG₁ (κ) class (subclass).

13. The anti-HCMV monoclonal antibody or its antigen binding portion according to claim 3, wherein the antibody belongs to the IgG₁ (κ) class (subclass).

14. A prophylactic or therapeutic agent for disease caused by the HCMV comprising the anti-HCMV monoclonal antibody according to claim 1.

15. A prophylactic or therapeutic agent for disease caused by the HCMV comprising the anti-HCMV monoclonal antibody or its antigen binding portion according to claim 2.

16. A prophylactic or therapeutic agent for disease caused by the HCMV comprising the anti-HCMV monoclonal antibody or its antigen binding portion according to claim 3.

17. The prophylactic or therapeutic agent for disease caused by the HCMV comprising: the anti-HCMV monoclonal antibody according to claim 1, wherein the disease caused by the HCMV is selected from any one of the following groups:
(a) interstitial pneumonia, retinitis, gastroenteritis and encephalitis with reactivation of HCMV in immunodeficiency state,
(b) cytomegalic inclusion disease caused by HCMV infection from pregnant mother to fetus,
(c) death due to spontaneous abortion and stillbirth caused by the cytomegalic inclusion disease, and death in the early years of life caused by the cytomegalic inclusion disease,
(d) when survived in the case of (c), microcephaly, mental development disorder, mental retardation and hearing impairment caused by the cytomegalic inclusion disease.

18. The prophylactic or therapeutic agent for disease caused by the HCMV comprising: the anti-HCMV monoclonal antibody or its antigen binding portion according to claim 2, wherein the disease caused by the HCMV is selected from any one of the following groups:
(a) interstitial pneumonia, retinitis, gastroenteritis and encephalitis with reactivation of HCMV in immunodeficiency state,
(b) cytomegalic inclusion disease caused by HCMV infection from pregnant mother to fetus,
(c) death due to spontaneous abortion and stillbirth caused by the cytomegalic inclusion disease, and death in the early years of life caused by the cytomegalic inclusion disease,
(d) when survived in the case of (c), microcephaly, mental development disorder, mental retardation and hearing impairment caused by the cytomegalic inclusion disease.

19. The prophylactic or therapeutic agent for disease caused by the HCMV comprising: the anti-HCMV monoclonal antibody or its antigen binding portion according to claim 3, wherein the disease caused by the HCMV is selected from any one of the following groups:
(a) interstitial pneumonia, retinitis, gastroenteritis and encephalitis with reactivation of HCMV in immunodeficiency state,
(b) cytomegalic inclusion disease caused by HCMV infection from pregnant mother to fetus,
(c) death due to spontaneous abortion and stillbirth caused by the cytomegalic inclusion disease, and death in the early years of life caused by the cytomegalic inclusion disease,
(d) when survived in the case of (c), microcephaly, mental development disorder, mental retardation and hearing impairment caused by the cytomegalic inclusion disease.

20. An isolated deoxyribonucleic acid (DNA) coding for an anti-HCMV monoclonal antibody or its antigen binding portion capable of binding to HCMV and neutralizing bioactivity of the HCMV, wherein the isolated DNA codes for any one of amino acid sequences of SEQ ID NOs. 1 to 4, three amino acid sequences of SEQ ID NOs. 5 to 7, or three amino acid sequences of SEQ ID NOs. 8 to 10.

21. An isolated DNA capable of hybridizing with the DNA described in claim 20 under stringent conditions.

22. A vector other than a plant expression vector, comprising the isolated DNA of claim 20.

23. A host cell other than a plant cell, comprising the vector, wherein the isolated DNA of claim 20 is integrated into the vector.


SEQUENCE LISTING (10 sequences)

SEQ ID NO: 1 - protein, 220 aa, Homo sapiens
DVVMTQSPLSLPVTLGQPASISCRSSESLVYSDGNTYLNWFQQRPGQSPRRLISKVSNRDSGVPDRFSGSGSGTDFTLKISRVEAEDVGVYYCMQGSHWPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, Homo sapiens
QVQLVESGGGMVQPGKSLRLSCAASGFTFSSYGMHWVRQAPGRGLEWVAIIWYDGDNTWYADSVKGRITISRDNSKNMLYLQMDSLRVEDTALYYCVRGGLGYFNGWDVDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 114 aa, Homo sapiens
DVVMTQSPLSLPVTLGQPASISCRSSESLVYSDGNTYLNWFQQRPGQSPRRLISKVSNRDSGVPDRFSGSGSGTDFTLKISRVEAEDVGVYYCMQGSHWPPYTFGQGTKLEIKR

SEQ ID NO: 4 - protein, 122 aa, Homo sapiens
QVQLVESGGGMVQPGKSLRLSCAASGFTFSSYGMHWVRQAPGRGLEWVAIIWYDGDNTWYADSVKGRITISRDNSKNMLYLQMDSLRVEDTALYYCVRGGLGYFNGWDVDHWGQGTLVTVSS

SEQ ID NO: 5 - protein, 16 aa, Homo sapiens
RSSESLVYSDGNTYLN

SEQ ID NO: 6 - protein, 7 aa, Homo sapiens
KVSNRDS

SEQ ID NO: 7 - protein, 10 aa, Homo sapiens
MQGSHWPPYT

SEQ ID NO: 8 - protein, 5 aa, Homo sapiens
SYGMH

SEQ ID NO: 9 - protein, 17 aa, Homo sapiens
IIWYDGDNTWYADSVKG

SEQ ID NO: 10 - protein, 13 aa, Homo sapiens
GGLGYFNGWDVDH